(19) United States
(12) Patent Application Publication
(10) Pub. No.: US 2014/0113276 A1
(43) Pub. Date: Apr. 24, 2014

(54) PRODUCTION PLANT FOR MICROALGAE BIOFUEL, BIOREACTOR FOR PRODUCING BIOFUEL, AND METHOD FOR PRODUCING MICROALGAE BIOFUEL

(71) Applicant: KOREA OCEAN RESEARCH AND DEVELOPMENT INSTITUTE, Gyeonggi-do (KR)

(72) Inventors: Do-Hyung Kang, Gyeonggi-do (KR); Soo-Jin Heo, Gyeonggi-do (KR); Chol-Hong Oh, Jeju-do (KR); Tae-Ho Kim, Gyonggi-do (KR); Heung-Sik Park, Seoul (KR); Affan Abu, Gyonggi-do (KR)

(21) Appl. No.: 14/119,414

(22) PCT Filed: Sep. 6, 2011
(86) PCT No.: PCT/KR2011/006578

§ 371 Date: Nov. 21, 2013

(30) Foreign Application Priority Data
May 25, 2011 (KR) 10-2011-0049544
May 25, 2011 (KR) 10-2011-0049545
May 27, 2011 (KR) 10-2011-0050438

(51) Int. Cl. C12M 1/00

(52) U.S. Cl. CPC C12M 21/12
USPC 435/3; 435/289.1; 435/292.1; 435/134; 435/286.1

(57) ABSTRACT

Disclosed is a plant for producing microalgae biofuel. The plant includes a plant space part having an internal space, a culture part provided in the internal space of the plant space part to culture microalgae by continuously circulating a fluid, which includes the microalgae and is supplied from an outside, at positions different from each other, and a temperature adjusting part to adjust a temperature value of the internal space in the plant space part to a temperature value in a preset temperature value range.

### TECHNICAL FIELD

The present invention relates to a plant for producing biofuel, and more particularly to a plant for producing a source material of biofuel and producing a microalgae biofuel by using the source material, in which the biofuel can be produced by making a growing environment to culture microalgae in large amounts in order to produce the biofuel.

The present invention relates to a bioreactor for producing biofuel, and more particularly to a bioreactor for producing biofuel, capable of culturing microalgae in large amounts by continuously and forcibly circulating a fluid containing microalgae and supplying oxygen.

The present invention relates to a method of producing a microalgae biofuel, and more particularly to a method of producing microalgae biofuel, capable of producing biofuel by culturing microalgae in large amounts.

### BACKGROUND ART

In general, microalgae are creatures living for a longest time on earth, and hundreds of thousands of species of microalgae are currently living. It is reported that only about 0.1% of the microalgae are bioactive microalgae, and only an extremely small amount of microalgae are cultivated in an industrial scale.

Representatively, microalgae, such as chlorella or spirulina, have been developed as various source materials for dietary supplements, health food supplements, feed fodders for aquiculture, alternative medicine, and energy resources. The microalgae are single-celled plants, which inhabit in fresh water or sea water and have no roots, stems, and leaves, and photosynthesize with chlorophyll. Since the microalgae contain plant fatty acid, protein, mineral, and a variety of vitamins, the microalgae are greatly utilized for health food. Since a large amount of microalgae can be obtained within the short time as the microalgae are rapidly grown and proliferated, the microalgae has infinite potential as source materials of the biodiesel.

In general, following two schemes are used to grow the microalgae by forming a culture environment of the microalgae.

First, microalgae and culture water are carried through a pipe so that the microalgae and the culture water are exposed to the sunlight as much as possible. As the microalgae are grown in a sealing-type environment similar to an experimental condition, the contamination of the microalgae is lowered. In addition, since equipment occupies a land smaller than that of an open type system, the productivity per hectare is highly represented.

However, conventionally, since a pipe having a length of several kilometers is required to produce an amount of oil in an industrial level, high-priced facilities are required, and the maintenance cost of the facilities is increased.

Second, in order to expose the microalgae to the sunlight, culture water must be flown out. In the case of the typical open type pond, the cost of facilities is lower than that in a closed type system. However, in the typical open type pond, stored seawater is kept in a sunken state, and the continuous supply of the oxygen is not performed, so that the microalgae may be perished.

In addition, since the open type pond is provided at an outdoor place, the peripheral temperature, as well as the temperature of seawater or fresh water including microalgae, is not controlled. Therefore, actually, the culture environment or the growing condition of the microalgae may not be variably adjusted.

### DISCLOSURE

### Technical Problem

An object of the present invention is to provide a plant for producing a source material of biofuel and producing microalgae biofuel by using the source material, capable of easily controlling a culture temperature to cultivate microalgae in large amounts and a growing condition such as photosynthesis.

Another object of the present invention is to provide a plant for producing microalgae biofuel, capable of easily growing microalgae by continuously circulating microalgae in a confined space and continuously supplying oxygen to the microalgae.

Still another object of the present invention is to provide a bioreactor for producing biofuel, capable of culturing microalgae in large amounts by storing a predetermined amount of fluid including microalgae, forcibly circulating the fluid, and continuously supplying oxygen, and lowering the cost of facilities.

Still yet another object of the present invention is to provide a bioreactor for producing biofuel, capable of adjusting a culture environment of a fluid including microalgae in real time.

Still yet another object of the present invention is to provide a method of producing microalgae biofuel, capable of culturing microalgae in large amounts by easily controlling growing conditions, such as a culture temperature and photosynthesis, and producing microalgae biofuel in large amounts.

Still yet another object of the present invention is to provide a method of producing microalgae biofuel, capable of easily growing microalgae by continuously circulating microalgae in a confined space of a plant and continuously supplying oxygen to the microalgae.

### Technical Solution

According to an aspect of the present invention, there is provided a plant for producing microalgae biofuel.

The plant includes a plant space part having an internal space, a culture part provided in the internal space of the plant space part to culture microalgae by continuously circulating a fluid, which includes the microalgae and is supplied from an outside, at positions different from each other, and a temperature adjusting part to adjust a temperature value of the internal space in the plant space part to a temperature value in a preset temperature value range.

The plant space part includes a bottom part including concrete, a wall part surrounding edges of the bottom part, a ceiling part to cover an upper portion of the plant space part to form the internal space from the edges of the wall part, and one or more doors installed in the wall part to open/close the internal space.

Further, preferably, each of the wall part, the ceiling part, and the doors includes lattice frames comprising aluminum (Al) and glass plates installed between the lattice frames.

In addition, the culture part includes a water tank provided in the internal space of the plant space part to store a predetermined amount of a fluid and having a circulation flow path of the fluid formed therein, and a water wheel provided in the water tank and rotated by receiving power from an outside to forcibly move the fluid along the circulation flow path.

Further, preferably, a lower portion of the water tank is inserted into the bottom part.

In this case, preferably, the water tank includes a water tank body having an open upper portion and having a storage space in which the fluid is stored, and a separator protruding upward from an internal floor of the water tank body to form the circulation flow path.

Preferably, inclined surfaces are formed on a lateral side of the storage space in the water tank body and an outer surface of the separator to guide an interval between the lateral side of the storage space of the water tank body and the outer surface of the separator such that the interval is gradually narrowed.

In addition, preferably, fluid confinement grooves are formed in a lateral side of the storage space of the water tank body and the separator to limit a storage level of the fluid.

In addition, preferably, the water wheel includes a support member selectively fixed to the water tank body such that the support member is provided on the circulation flow path, a rotational shaft having both end portions rotatably supported on the support member such that the rotational shaft receives power from an outside to rotate, first blades having a plate shape and installed radially on the rotational shaft, second blades installed on end portions of the first blades and inclined at a predetermined angle with respect to each first blade, a rotational motor linked with the rotational shaft to rotate the rotational shaft, and a water wheel control unit to control an operation of the rotational motor.

Further, preferably, the water wheel control unit receives an electrical signal from the temperature adjusting part to variably adjust a rotational speed of the rotational shaft based on the temperature value.

The temperature adjusting part includes a heating unit installed on a floor of the culture part to heat the floor to a predetermined temperature, one or more open/close units to receive an electrical signal from an outside to open/close the internal space of the plant space part, a temperature sensor to measure an internal temperature value of the plant space part, and a control unit to control operations of the heating unit and the open/close unit such that the measured temperature value by the temperature sensor is in the preset reference temperature value range.

Preferably, the open/close unit includes an open door rotatably supported on the plant space part to open/close an inner part of the plant space part, a motor installed in the plant space part, and having a motor shaft to receive a control signal from the control unit to rotate, a gear linked with the motor shaft, and a rack protruding from the open door and engaged with the gear along a path having a predetermined curvature to rotate the open door according to a rotational motion of the motor shaft.

In addition, preferably, the heating unit includes circulation pipes buried in the floor of the culture part while being spaced apart from each other by a predetermined distance, and a boiler linked with the circulation pipes to heat heating water to a predetermined temperature by receiving an electrical signal from the control unit and to supply the heating water to the circulation pipes such that the heating water is circulated.

Besides, the plant further includes a lighting part installed in the plant space part.

In this case, preferably, the lighting part includes a light emitting device provided at an upper portion of the culture part to receive an electrical signal from an outside and to form predetermined illuminance, and a lighting control unit electrically connected with the light emitting device to transmit a signal to the light emitting device such that preset illuminance and preset time to emit light are accomplished.

Meanwhile, there is provided a method of producing microalgae biofuel.

The method includes storing a fluid including the microalgae in a bioreactor which has a circulation flow path formed therein and is surrounded in a plant, continuously circulating the fluid along the circulation flow path, adjusting a temperature value of an internal space in the plant to a temperature value in a preset temperature value range, or controlling a temperature value of the bioreactor according to the temperature value of the internal space in the plant, and separating microalgae from the fluid and pressing an oil.

In this case, preferably, the circulating of the fluid along the circulation flow path includes providing a forcible circulator, which forcibly forms a flow on the circulation path, in the bioreactor, and forming continuous and forcible flow of the fluid along the circulation flow path by actuating the forcible circulator.

In addition, preferably, the bioreactor is provided therein with a separator to form the circulation flow path, level confinement grooves are formed in sidewalls and the separator of the bioreactor, and the fluid is stored in the bioreactor by forming a level of the fluid to the level confinement grooves.

In addition, preferably, the continuously-circulating of the fluid along the circulation flow path includes controlling an operation of the forcible circulator such that a forcible flow speed is varied depending on the temperature value of the internal space in the plant.

In addition, the adjusting of the temperature value of the internal space in the plant includes adjusting a temperature of the bioreactor depending on the temperature value of the internal space in the plant, and adjusting ventilation in the plant depending on the temperature value of the internal space in the plant.

In this case, the adjusting of the temperature of the bioreactor includes measuring the temperature value of the internal space in the plant by using a temperature sensor, transmitting the measured temperature value of the internal space in the plant to a control unit, and hating a floor of the bioreactor to a predetermined temperature by actuating a heating unit installed in the floor of the bioreactor through the control unit, such that the temperature value measured by the temperature sensor is in a range of a preset first reference temperature value range.

Further, preferably, a circulation pipe is buried in the floor of the bioreactor, heating water is heated to a predetermined temperature by using a boiler which receives an electrical signal from the control unit, and the heating water, which is heated, is supplied into the circulation pipe and circulated to heat the floor of the bioreactor.

In addition, preferably, the temperature value of the internal space in the plant is measured by using a temperature sensor and transmitted to a control unit, and the internal space in the plant is open/closed by actuating one or a plurality of ventilation adjusting units using the control unit such that the temperature value measured by the temperature sensor is in a preset second reference temperature value range.

Further, the method further includes constantly providing light to the fluid circulated along the circulation flow path.

Preferably, the constantly providing of the light includes arranging lamps on an upper portion of the bioreactor such that the lamps receive an electrical signal from an outside and emit light with predetermined illuminance, and accomplishing preset illuminance and preset time to emit light by using a light control unit.

### Advantageous Effects

As described above, according to the present invention, the growing conditions, such as a culture temperature and photosynthesis, to cultivate the microalgae in large amounts can be easily controlled.

In addition, according to the present invention, the microalgae can be easily grown by continuously circulating the microalgae in a confined space and continuously supplying oxygen to the microalgae.

Further, according to the present invention, the microalgae can be cultivated in large amounts and the microalgae biofuel can be produced in large amounts by easily controlling the growing condition such as the culture temperature and the photosynthesis.

In addition, according to the present invention, the microalgae can be easily grown by continuously circulating the microalgae in an internal confined space of the plant and continuously supplying oxygen to the microalgae.

### DESCRIPTION OF DRAWINGS

FIG. 1 is a front view showing a plant for producing microalgae biofuel according to the present invention.

FIG. 2 is a rear view showing the plant for producing the microalgae biofuel according to the present invention.

FIG. 3 is a side view showing the plant for producing the microalgae biofuel according to the present invention.

FIG. 4 is a sectional view showing the plant for producing the microalgae biofuel according to the present invention.

FIG. 5 is a plan view showing a plant space part according to the present invention.

FIG. 6 is a sectional view showing a water tank according to the present invention.

FIG. 7 is a partial enlarged sectional view showing a marked part A of FIG. 6.

FIG. 8 is a plan view showing the water tank according to the present invention.

FIG. 9 is a perspective view showing a water wheel of FIG. 8.

FIG. 10 is a perspective view showing the state that the water wheel of FIG. 8 is installed in a body of the water tank.

FIG. 11 is a plan view showing a ceiling part of the plant space part according to the present invention.

FIG. 12 is a view showing an open/close unit according to the present invention.

### BEST MODE

### Mode for Invention

Hereinafter, a plant for producing microalgae biofuel according to the present invention will be described with reference to accompanying drawings.

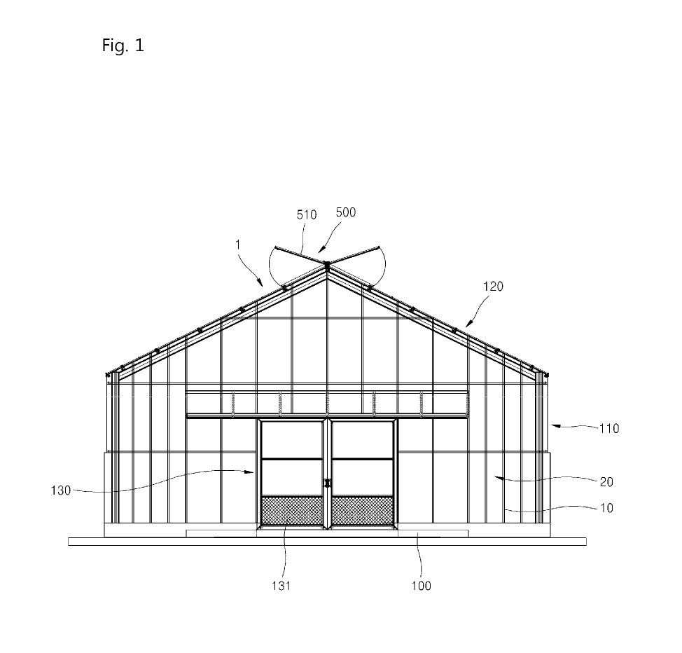

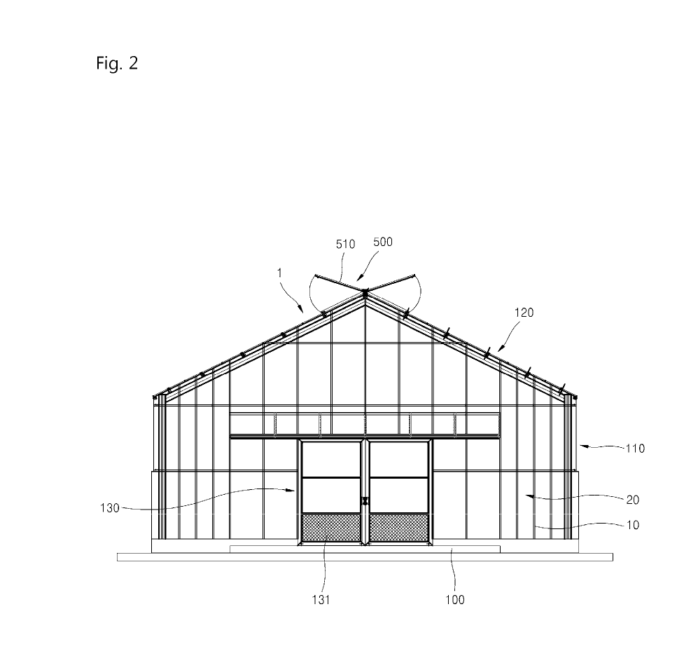

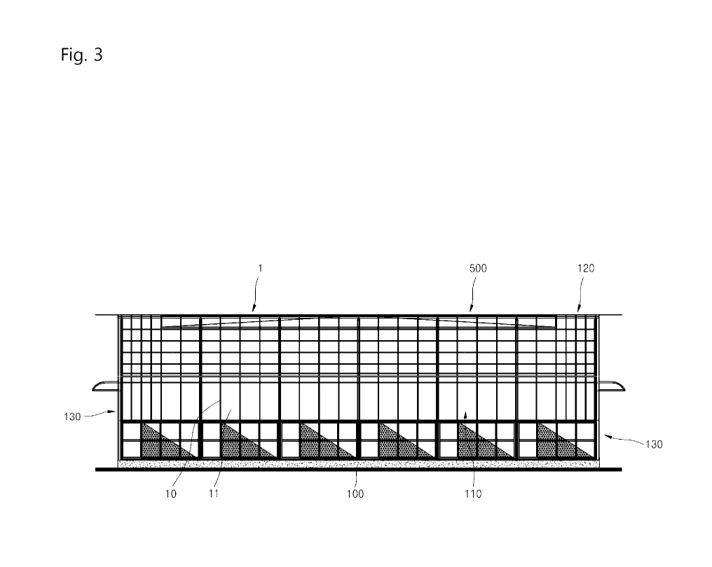

FIGS. 1 and 2 are a plan view and a rear view showing the plant for producing the biofuel according to the present invention. FIG. 3 is a side view showing an internal space of the plant of FIGS. 1 and 2.

Referring to FIGS. 1 to 3, the plant for producing the biofuel according to the present invention mainly includes a plant space part 1, a culture part 2, and a temperature adjusting part.

Hereinafter, the plant space part 1 will be described.

The plant space part 1 includes a bottom part 100, a wall part 110, a ceiling part 120, and doors 130.

The bottom part 100 includes concrete.

The wall part 110 includes walls formed at a predetermined height upward from the edges of the bottom part 100.

The wall part 110 includes lattice frames 10. The lattice frames 10 include a metallic material such as aluminum. The aluminum includes soft metal and can radiate heat to the outside.

Glass plates 20 are installed between the lattice frames 10. The glass plates 20 include a transparent material. The glass plates 20 transmit the external sun light into the internal space.

The ceiling part 120 is arranged to cover an upper portion of the well part 110. The ceiling part 120 has the substantially same components as those of the wall part 110. The upper most end portion of the ceiling part 120 is pointed.

The ceiling part 120 is provided at the upper most end portion thereof with open/close units 500. The components and the operation of the open/close units 500 will be described below.

As shown in FIGS. 1 and 2, the doors 130 are installed in front and rear surface portions of the plant space part 1. Each door 130 includes lattice frames 10 the same as those of the wall part 110, and glass plates 11 installed between the lattice frames 10.

In addition, a net 131, such as an insect net, which is provided in the form of a mesh having a uniform size, is additionally installed in each door 130. The net 131 may be used as an inflow passage of an outdoor air. In addition, the net 131 may be used for the purpose of preventing harmful insects from being invaded into the plant space part 1.

Hereinafter, the culture part 2 according to the present invention will be described.

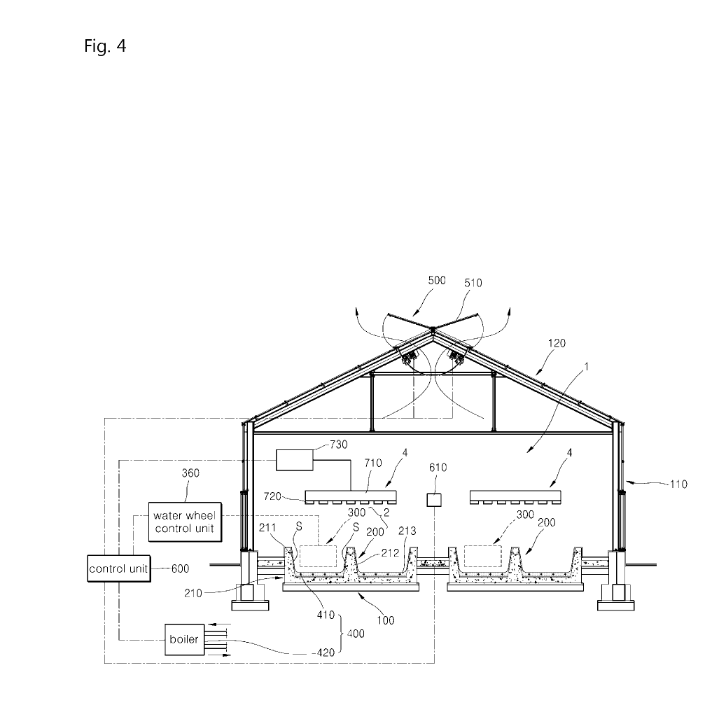

FIG. 4 is a sectional view showing the plant space part.

Referring to FIG. 4, the culture part 2 is installed at the bottom part 100 of the plant space part 1.

The culture part 2 includes one or more water tanks 200 and a plurality of water wheels 300 installed in each water tank.

As shown in FIG. 4, a pair of the water tanks 200 may be installed in parallel. In this case, one water tank 200 has the same components as those of the other water tank 200. Accordingly, the components of only one water tank 200 will be described.

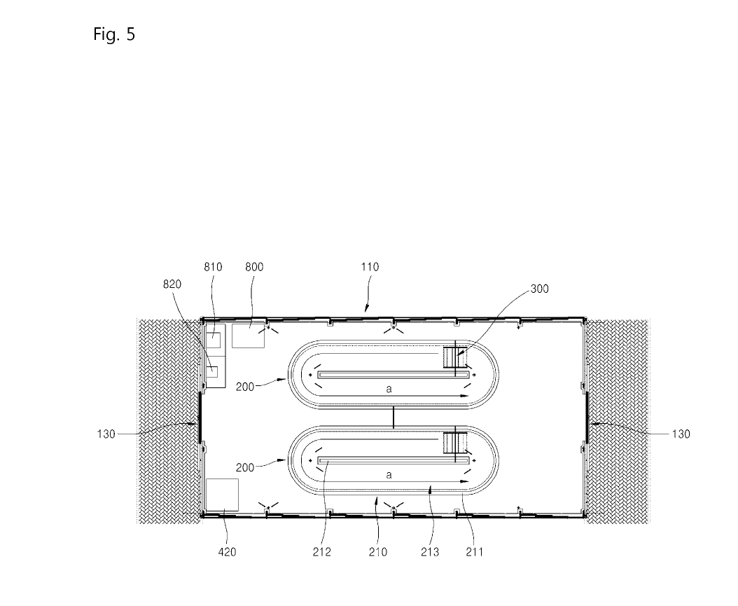

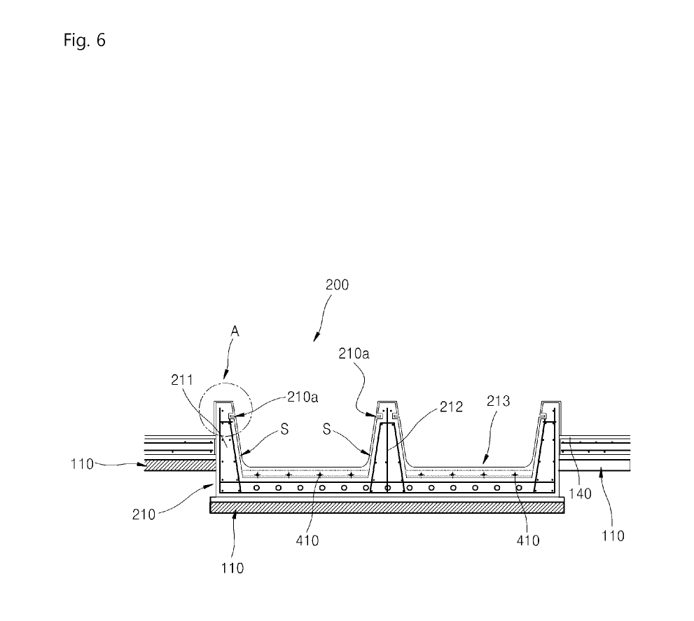

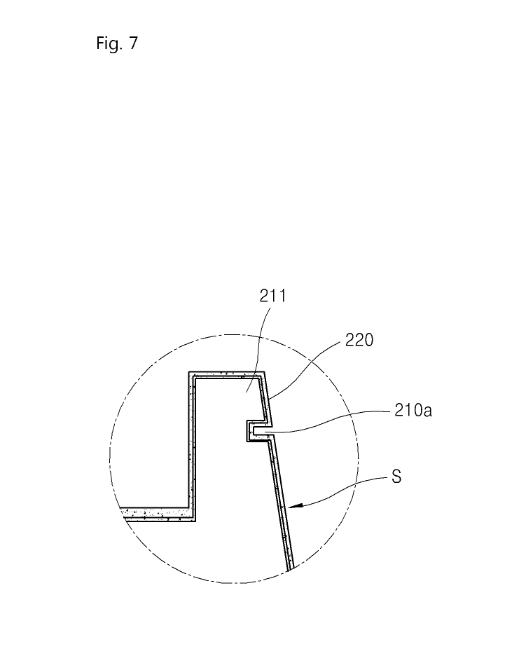

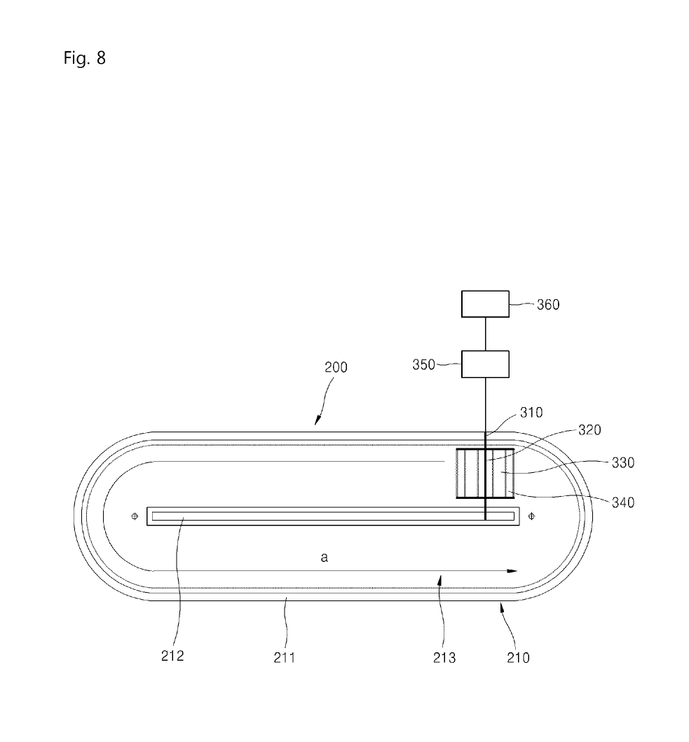

FIG. 5 is a view showing the arrangement state of a pair of water tanks. FIG. 6 is a sectional view showing the water tank. FIG. 7 is a view showing a marked part A of FIG. 6. FIG. 8 shows a flat surface of the water tank.

Referring to FIG. 4, the water tank 200 includes a water tank body 210 and a separator 212 formed at the central portion of the water tank body 210.

The water tank body 210 includes a water tank floor 213 and sidewalls 211 extending upward from edges of the water tank floor 213. The storage space of the water tank 200 is surrounded by the water tank floor 213 and the sidewalls 211, and the upper portion of the storage space is exposed to the outside.

The water tank body 210 is connected with a fluid supply part (not shown), which supplies a fluid including microalgae, through a pipe. The storage space of the water tank body 210 may contain therein a predetermined amount of a fluid received from the fluid supply part.

The water tank body 210 includes concrete in which steel bars are arranged.

In addition, a lower portion of the wart tank body 210 is partially inserted into the bottom part 100 of the plant space part 1. Accordingly, the water tank body 210 may receive a predetermined amount of geothermal heat from the bottom part 100 through the lower portion of the water tank body 210.

The separator 212 is formed at the central portion of the water tank body 210.

The separator 212 has a predetermined length in a longitudinal direction of the water tank body 210 and has a predetermined height upward. The height of the separator 212 is substantially equal to that of the sidewall 211 of the water tank body 210.

As the separator 212 is installed, a circulation flow path a is formed in the storage space of the water tank body 210 to circulate a fluid as shown in FIG. 5.

Referring to FIG. 6, the interval between the sidewall 211 and the separator 212 of the water tank body 210 is gradually narrowed downward.

Inclined surfaces S are formed on a lateral side of the sidewall 211 and an outer surface of the separator 212, respectively. Each inclined surface S forms an obtuse angle with respect to the water tank floor 213 of the water tank body 210.

In addition, the water tank body 210 is provided therein with fluid confinement grooves 210a.

The fluid confinement grooves 210a are formed in the sidewalls 211 of the water tank body 210 and the separator 212.

Each fluid confinement groove 210 is formed at a predetermined installation height from the water tank floor 213 of the water tank body 210.

The fluid confinement groove 210a formed in the sidewall 211 at the side of the storage space of the water tank body 210 is seamlessly formed along the outer surface of the side wall 211 while maintaining the same installation height.

The fluid confinement groove 210a is seamlessly formed in the outer surface of the separator 212 at the installation height.

Therefore, the fluid confinement grooves 210a formed in the separator and the sidewall are formed at the same installation height.

As shown in FIG. 7, the fluid confinement groove 210a may be a rectangular groove, or a groove having a curved inner peripheral surface.

Therefore, the level of the fluid stored in the storage space of the water tank body 210 may be uniformly confined by the fluid confinement groove 210a.

In addition, referring to FIG. 7, a water proof layer 220 is formed with a predetermined thickness on an outer surface of the water tank body 210. The water proof layer 220 is a layer including a PVC liner.

In addition, as shown in FIG. 6, another water proof layer 140 is formed even on the plant space part 1 having the bottom part 100 into which the lower portion of the water tank body 210 is partially inserted.

Referring to FIGS. 5 and 8, the water wheels 300 are installed in the water tank body 210.

The water wheel 300 is installed on the circulation flow path a formed in the water tank body 210.

The circulation flow path a includes two linear flow paths formed at both sides of the separator 212 and two curved flow path linking both end portions of the two linear flow paths with each other.

The water wheel 300 is preferably arranged at the boundary between the linear flow path and the curved flow path.

Hereinafter, the structure of the water wheel 300 will be described with reference to FIGS. 8 to 10.

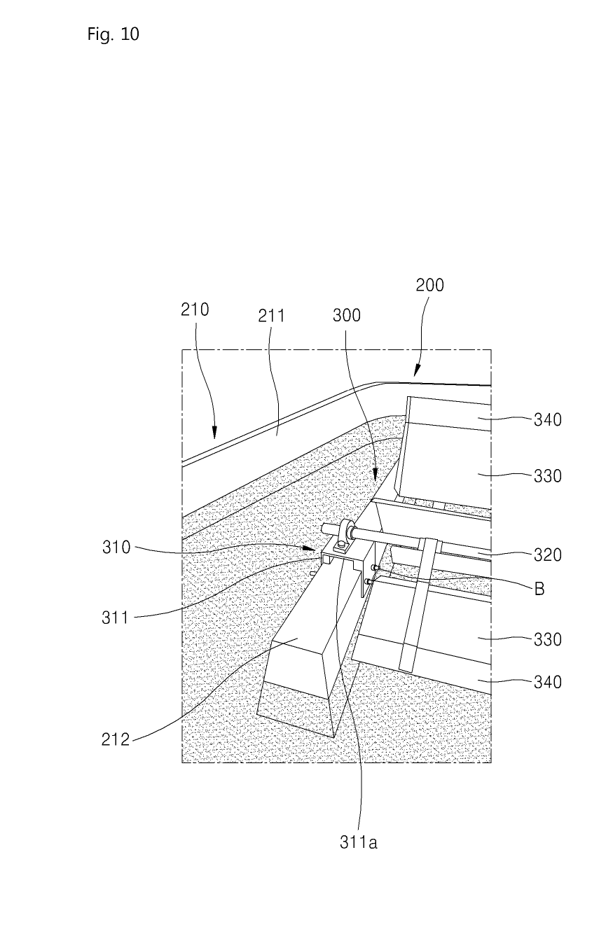

Referring to FIGS. 8 and 10, the water wheel 300 has a pair of support members 310.

The support members 310 includes a support plate 311 provided therein with a support groove 311a open upward.

The support groove 311a of the support plate 311 provided in each support member 310 is fitted around the upper end portion of the sidewall 211 or the upper end portion of the separator 212. In this state, the support plate 311 of each support member is coupled with the upper end portion through a bolt B.

Referring to FIGS. 8 and 10, both end portions of a rotational shaft 320 is rotatably supported by the paired support members 310.

A rotational motor 350 is coupled with one end portion of the rotational shaft 320.

A water wheel control unit 360 shown in FIG. 8 is electrically connected with the rotational motor 350 to control the operation of the rotational motor 350.

A first blade 330 has corrosion resistance.

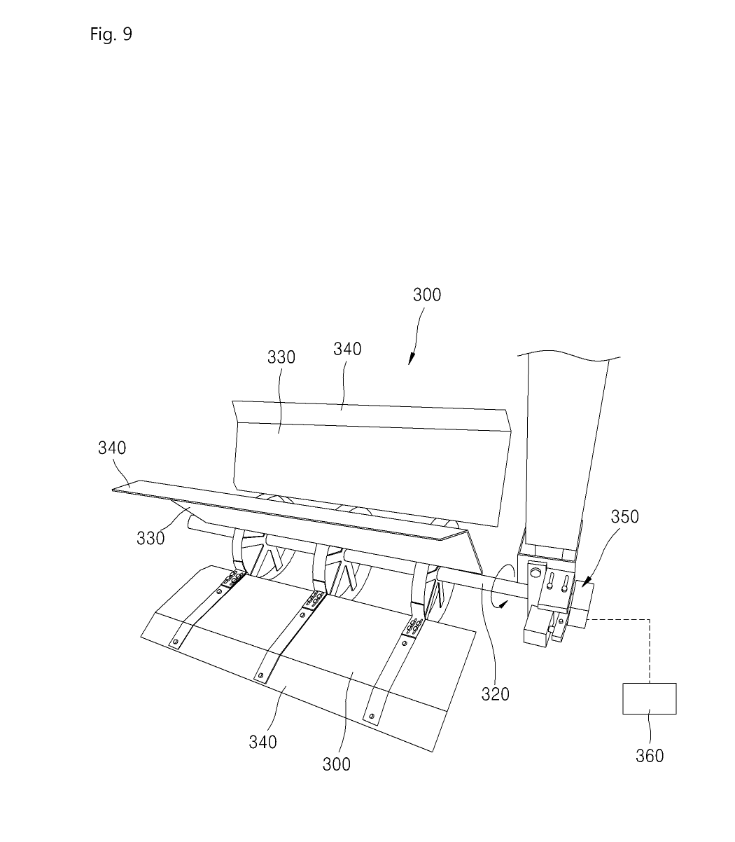

Referring to FIGS. 9 and 10, one end portion of the first blade 330 is fixed to the rotational shaft 320. The first blade 330 is installed radially from the rotational shaft 320.

A second blade 340 is installed at the end portion of the first blade 330. The second blade 340 is inclined from the first blade 330 at a predetermined degree.

The first and second blades 330 and 340 are arranged in such a manner that the first and second blades 330 and 340 are sequentially impregnated into the fluid stored in the storage space of the water tank body 210 as the above end portions of the first and second blades 330 and 340 rotate.

As the second blade 340 inclined from the first blade 330 is rotated, the second blade 340 may forcibly move the fluid along the circulation flow path a. In addition, the second blade 340 may draw up the fluid and drop down the drawn-up fluid at a predetermined height.

Therefore, the water wheel 330 forcibly forms the circulation flow path a in the water tank body 210 by using the first and second blades 330 and 340, which are rotated, draws up a circulating fluid, and drops down the fluid, so that oxygen can be continuously generated in the circulating fluid.

Hereinafter, a temperature adjusting part according to the present invention will be described.

Referring to FIG. 4, the temperature adjusting part includes a heating unit 400, a plurality of open/close units 500, a temperature sensor 610, and a control unit 600.

The heating unit 400 includes a circulation pipe 410 and a boiler 420.

The circulation pipe 410 is buried in the lower portion of the water tank body 210. The circulation pipe 410 is provided in zigzags. Both end portions of the circulation pipe 410 are coupled with the boiler 420. Heating water is introduced into one end portion of the circulation pipe 410, and discharged from the other end portion of the circulation pipe 410.

The boiler 420 is electrically connected with the control unit 600. The control unit 500 controls the operation of the boiler 420.

The boiler 420 receives an electrical signal from the control unit 600 and heats heating water, which is supplied from the outside, to a predetermined temperature so that the heating water is supplied to the circulation pipe 410. The heating water supplied to the circulation pipe 410 circulates along the circulation pipe 410.

Accordingly, the heating unit 400 may heat the floor of the water tank body 210 to a predetermined temperature.

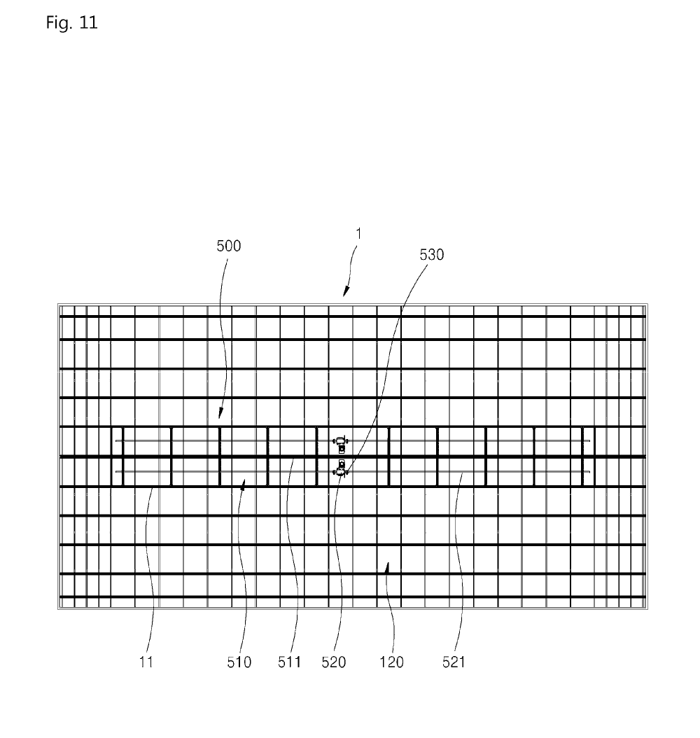

FIG. 11 shows the ceiling part 120 of the plant space part 1.

Referring to FIG. 11, the open/close units 500 are installed in the ceiling part 120 of the plant space part 1.

Two open/close units 500 are provided, and installed symmetrically to each other at both sides about the upper end portion of the ceiling part 120.

The ceiling part 120 is provided therein with an opening 11 in which the two open/close units 500 are installed.

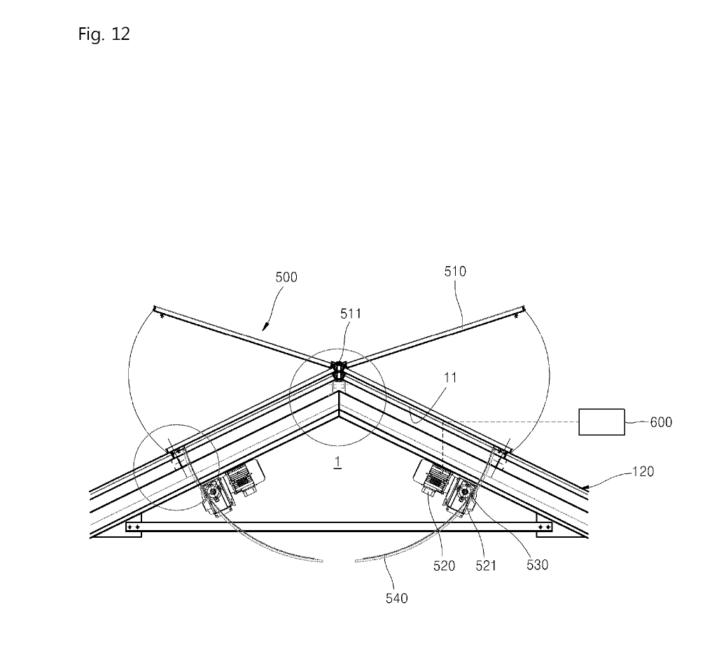

FIG. 12 shows the open/close units 500 according to the present invention.

Hereinafter, the structure of one open/close unit 500 will be described.

Referring to FIG. 12, each open/close unit 500 includes an open door 510 having a hinge shaft 511, a motor 520, a gear 530, and a rack 540.

The open door 510 is provided in the opening 11 formed in the ceiling part 120 of the plant space part 1.

The open door 510 has a predetermined width and a predetermined width so that the open door 510 is provided in the opening 11. The open door 510 may include lattice frames including metal such as aluminum, and a glass plate transparently installed between the lattice frames.

The hinge shaft 511 is installed at the boundary of the ceiling part 120.

Accordingly, the open door 510 is rotatable up and down about the hinge shaft 511.

The motor 520 is electrically connected with the control unit 600.

The motor 520 is fixedly installed in the lattice frame of the ceiling part 120 adjacent to the opening 11.

The motor 520 is fixed by an additional fixing bracket (not shown).

The motor 520 has a motor shaft 521 to receive a control signal from the control unit 600 and to rotate at a predetermined rotational speed. The motor shaft 521 is spaced apart from the hinge shaft 511 by a predetermined distance, and provided in parallel to the hinge shaft 511.

The motor shaft 521 is connected with the center of the gear 530. The gear 530 is formed in the shape of a disc.

The gear 530 is provided at the outer circumferential surface thereof with gear teeth. In this case, the gear is fixedly installed to the lattice frame adjacent to the opening 11 through an additional bracket (not shown).

In addition, the open door 510 is provided on the bottom surface thereof with the rack 540 having a predetermined length and the shape of “U”.

The rack 540 forms a path having a curvature, and is geared with the gear teeth of the gear 530.

The rotation of the gear 530 guides the forcible movement of the rack 540. The rack 540 forcibly performs a rotational motion up and down. The open door 510 may rotate up and down since the end portion of the rack 540 is fixed to the bottom surface of the open door 510.

As described above, according to the present invention, two open/close units 500 having the above structure are provided. In addition, the two open/close units 500 are provided symmetrically to each other about the boundary of the ceiling part 120.

The control unit 500 may control the open doors 510 of the open/close units 500 so that the open doors 510 of the open/close units 500 simultaneously or independently rotate.

Referring to FIG. 4, the temperature sensor 610 is electrically connected with the control unit 600.

The temperature sensor 610 may be a device such as a thermocouple, which measures the temperature value of the internal space of the plant space part 1 and transmits the measured temperature value to the control unit 600.

Preferably, the temperature sensor 610 may be arranged in such a manner that the temperature sensor 610 measures the temperature values of the upper space of the water tank 200 and the space surrounding the water tank 200.

As described above, the control unit 600 is electrically connected with the motor 520 of the open/close unit 500 and the temperature sensor 610.

A reference temperature value range is set in the control unit 600. The reference temperature value range may be variably set in the control unit 600 by a device such as an input device (not shown).

For example, the reference temperature value range may be in the range of 20° C. to 30° C. The reference temperature value range allows microalgae to be easily cultivated.

Referring to FIG. 5, the plant according to the present invention includes a lighting part 4.

The lighting part 4 includes light emitting devices 720, a base 710 to fix the light emitting devices 720 thereto, and a lighting control unit 730.

The lighting part 4 is provided at the upper portion of each water tank 200.

The base 710 may be provided at the upper portion of the water tank 200, and may be fixed by a fixing bar (not shown) extending from the ceiling part 120.

The light emitting devices 720 are provided on the base 710.

The lighting control unit 730 controls the operation of the light emitting devices 720.

The time to emit light and the illuminance of the light of the light emitting devices 720 are preset in the lighting control unit 730. Accordingly, the lighting control unit 730 controls the operation of the light emitting devices 720 so that the preset time to emit the light and the preset illuminance of the light can be accomplished. For example, the time to emit light may be in the range of 17:00 p.m. to 22:00 p.m.

The light emitted from the light emitting device 720 is irradiated to the fluid stored in the water tank body 210. In the case of the fluid including microalgae, the microalgae may perform photosynthesis due to the light irradiated thereto.

The plant according to the present invention includes a centrifuge 800 and an oil press 810.

The centrifuge 800 and the oil press 810 are provided at the internal space of the plant part 1.

The centrifuge 800 is linked with the water tank body 210 through a pipe (not shown). The pipe is provided thereon with a valve (not shown) to open/close a flow path. The pipe is provided thereon with a pump (not shown) to pump the fluid.

If the valve is open and the pump operates, the fluid including the microalgae circulating in the storage space of the water tank body 210 is moved to the centrifuge 800 through the pipe.

The centrifuge 800 may separate an oil component of the fluid from other components of the fluid having the specific gravity different from that of the oil component.

The oil press 810 is linked with the centrifuge 800 through a pipe (not shown).

The pipe between the centrifuge 800 and the oil press 810 is provided thereon a pump (not shown) to pump the fluid from the pipe.

The oil press 810 receives the oil component, which is separated by the centrifuge 800, through the pipe, and excludes other components from the oil component to press biodiesel.

In addition, the pressed biodiesel is stored in an additional storage unit 820 along a discharge line.

Hereinafter, the operation of the plant for producing biofuel according to the present invention having the above structure will be described.

Referring to FIGS. 1 to 3, since the plant space part 1 according to the present invention is constructed by using the lattice frame 10 including an aluminum material, the plant space part 1 can be easily installed and radiate heat to the outside.

Further, the glass plates 20 installed between the lattice frames 10 allow external sun light to be easily transmitted into the internal space.

Therefore, the plant space part 1 has the atmosphere such as a greenhouse.

The two doors 130 installed at both side portions of the plant space part 1 guide the access of a worker to the plant space part 1. In addition, since the two doors 130 face each other, the ventilation in the internal space can be smoothly accomplished.

The open/close unit 500 installed at the upper portion of the plant space part 1, preferably, the ceiling part 120 receives an electrical signal from the outside to perform an open/close operation. The ventilation of the internal space and the temperature adjustment can be performed through the open/close operation, and the details thereof will be described in detail.

Referring to FIGS. 4 and 5, two water tanks 200 are installed in the internal space of the plant space part 1.

One of the two water tanks 200 may store seawater therein, and the other may store fresh water therein. In this case, the type of the fluid stored in each water tank 200 may be selectively varied.

Hereinafter, one water tank 200 will be representatively described.

A predetermined of seawater including microalgae is stored in the storage space of the water tank body 210. The seawater may be stored in the storage space of the water tan body 210 through a fluid supply unit (not shown).

The separator 212, which is formed with a predetermined length at the central portion of the water tank floor 213 of the water tank body 210, is spaced apart from the sidewall 211 by a predetermined distance to form the circulation flow path a of the fluid in the storage space of the water tank body 210. The circulation flow path a includes a linear flow path and a curved flow path.

In this case, it is preferred that the level of the seawater stored in the water tank body 210 is confined by the fluid confinement groove 210a.

Referring to FIGS. 6 and 7, the fluid confinement grooves 210a are formed at the same height in the outer circumferential surfaces of the sidewall 211 and the separator 212 of the water tank body 210.

Therefore, the seawater is stored in the storage space of the water tank body 210 at the level equal to the height at which the fluid confinement groove 210a is located.

Referring to FIGS. 8 and 9, the water wheel is provided on the circulation flow path a of the water tank body 210. In this case, the water wheel is preferably installed in the water tank 200 in such a manner that the second blades 340 are sequentially impregnated into the seawater while rotating.

The water wheel control unit 360 drives the rotational motor 350. The rotational motor 350 rotates the rotational shaft 320 at a predetermined rotation speed.

Accordingly, the first blades 330 formed radially on the outer circumference of the rotational shaft 320 rotate at a predetermined rotational speed. In addition, the second blades 340 inclined from the end portion of the first blade 330 simultaneously rotate.

The second blades 340 may circulate the fluid along the circulation flow path a while rotating.

The second blades 340 may sequentially draw up the seawater stored in the water tank body 210 while rotating. In addition, the seawater, which has been drawn up to a predetermined upper position, may be dropped down.

A procedure of drawing up the seawater and dropping down the drawn-up seawater for the purpose of storage according to the rotation of the second blades 340 is repeated.

The seawater may circulate along the circulation flow path a of the water tank body 210 through the repeated procedure.

The seawater, which is drawn up by the rotation of the second blade 340, is dropped down at a predetermined position so that the seawater is stored in the water tank body 210. In this case, bubbles may be formed in the seawater of the water tank body 210 due to the head, and oxygen may be supplied to the seawater.

The seawater circulating along the circulation flow path a in the water tank body 210 may receive oxygen through the continuous dropping procedure.

Therefore, microalgae included in the seawater continuously circulate while receiving oxygen, so that the microalgae can be easily grown.

The water tank 200 includes concrete in which steel bars are arranged, and is provided on an outer surface thereof with the water proof layer 220 as shown in FIG. 7. The seawater or the freshwater stored in the water tank body 210 can be prevented from leaking to the outside, or prevented from being infiltrated into the water tank body 210 due to the water proof layer 220.

Referring to FIG. 6, the inclined surfaces S are formed on the inner lateral side of the sidewall 211 of the water tank body 210 and formed on the outer lateral side of the separator 212, respectively. The width of the circulation flow path a of the water tank 200 is gradually reduced downward when viewed in a sectional view.

The seawater stored in the storage space of the water tank body 210 can be stably circulated due to the inclined surface S, so that the seawater can be prevented from overflowing out of the storage space when circulating.

Referring to FIG. 4, the lighting part 4 according to the present invention may cause the microalgae, which are included in the seawater circulating in the water tank 200, to perform photosynthesis.

The light emitting devices 720 of the lighting part 4 are provided at the upper portion of the water tank body 210 in the state that the light emitting devices 720 are installed on the base 710.

The lighting control unit 730 electrically connected to the light emitting devices 720 may control the light emission of the light emitting devices 720 in such a manner that the light emitting devices 720 emits light with the preset illuminance in a preset time range.

For example, if the time to emit light is set in the range of 17:00 p.m. to 22:00 p.m., the lighting control unit 730 controls the light emission of the light emitting devices 720 in such a manner that the light emitting devices 720 emit light with the preset illuminance in the preset time range.

The light emitted from the light emitting devices 720 is supplied to the seawater circulating along the circulation flow path a of the water tank body 210.

The microalgae included in the seawater may perform photosynthesis by the light supplied from the outside.

The light emission area of the light emitted from the light emitting devices 720 according to the present invention may be set to include a top surface area of the storage space of the water tank body 210, or may be set to include the top surface area of the circulation flow path a.

In addition, the light emission area may be set to include a portion of the upper region of the circulation flow path a.

The light emission area may be adjusted based on an area of the base 710 in which the light emitting devices 720 are installed. Alternatively, the light emission area may be adjusted by rotatably installing the base 710 at the upper portion of the water tank body 210 to adjust a light emission angle.

The lighting part 4 according to the present invention provides an environment of causing the microalgae, which are stored in the water tank body 210 and included in the seawater, to perform photosynthesis.

Although not shown in drawings, the light emitting devices 720 may be installed on the first and second blades 330 and 340 of the water wheel 300 shown in FIG. 9. In this case, the light emitting devices 720 are preferably installed on the first and second blades 330 and 340 in the state that the light emitting devices 720 are surrounded by a water proof material (not shown). Naturally, the light emitting devices 720 may be installed on the rotational shaft 320 of the water wheel 300.

In addition, although not shown in drawings, the light emitting devices 720 shown in FIG. 4 may be installed in the sidewall 211, the separator 212, and the water tank floor 213 of the water tank body 210.

In this case, the light emitting devices 720 may directly provide light to the seawater stored in the storage space of the water tank body 210.

Referring to FIG. 4, the temperature adjusting part 3 according to the present invention may adjust a temperature value of the internal space in the plant space part 1 to a temperature value in the preset temperature value range.

The temperature sensor 610 measures the temperature value of the internal space in the plant space part 1 in real time. The temperature sensor 610 transmits the measured temperature value to the control unit 600.

The control unit 600 determines if the measured temperature value is in the preset temperature value range. For example, the preset temperature value range may be the range of 20 to 30.

In addition, the control unit 600 controls the operations of the open/close units 500 and the heating unit 400 so that the measured temperature value is included in the preset temperature value range.

Hereinafter, the operations of the open/close units 500 will be described. In this case, the operation of one open/close unit 500 will be representatively described below.

Referring to FIGS. 11 and 12, the open door 510 makes the opening 11, which is formed in the ceiling part 120, closed.

The control unit 600 actuates the motor 520, and the motor 520 rotates the motor shaft 521 in one direction.

The gear 530 linked with the motor shaft 521 rotates in one direction in cooperation with the motor shaft 521.

The gear 530 is toothed with one surface of the rack 540 having the shape of “U” and installed on the bottom surface part of the open door 510.

Accordingly, as the gear 530 rotates in one direction, the rack 540 cooperating with the gear 530 is lifted upward, that is, the direction in which the open door 510 is open. In addition, the open door 510 linked with the rack 540 opens the opening 11 of the ceiling part 120.

Further, the other open/close unit 500 performs the same operation as that of one open/close unit 500.

The opening 11 of the ceiling part 120 is opened by the open door 510 that is opened, and the internal space of the plant space part 1 is exposed to the outside by the opening 11 that is opened.

Therefore, the air may be introduced into the internal space of the plant space part 1 through the opening 11.

In the case of summer, since the temperature of the air may be lower than the internal temperature of the plant space part 1, the internal temperature of the plant space part 1 may be dropped to a predetermined value or less as the air is introduced into the internal space of the plant space part 1.

Further, the control unit 600 controls the operation of two open/close units 500.

In this case, the control unit 600 may simultaneously actuate the motors 520 of two open/close units 500, or may independently actuate the motors 520 of the two open/close units 500.

Therefore, the control unit 500 may simultaneously open/close the open doors 510, or only one door. Accordingly, an amount of the air introduced into the internal space of the plant space part 1 may be adjusted.

Hereinafter, the operation of the heating unit 400 will be described.

Referring to FIG. 4, the control unit 600 actuates the boiler 420.

The boiler 420 receives heating water from the outside. The boiler 420 heats the heating water to a predetermined temperature, and then supplies the heating water to the circulation pipe 410.

Both end portions of the circulation pipe 410 are linked with the boiler 420, and the heating water supplied into the circulation pipe 410 may circulate along the circulation pipe 410.

In this case, the circulation pipe 410 is buried in the floor of the water tank body 210.

The circulation pipe 410 in which the heating water heated to the predetermined temperature is circulated is heated to a predetermined temperature. The heated temperature is transmitted to the floor of the water tank body 210.

Therefore, the storage space of the water tank body 210 and the seawater stored in the storage space may be raised to a predetermined temperature.

Further, the floor of the water tank body 210 is partially inserted into the bottom part 100 of the plant space part 1.

Therefore, in the case of winter, the water tank body 210 may directly receive geothermal heat through the water tank floor 213 of the water tank body 210.

As described above, the temperature adjusting part controls the operations of the open/close units 500 and the heating unit 400 in real time, so that the temperature value of the internal space in the plant space part 1 is constantly in the range of 20 to 30 throughout four seasons.

Meanwhile, the temperature adjusting part may control the operation of the water wheel 300 shown in FIGS. 9 and 10.

Referring to FIGS. 4, 9, and 10, the water wheel control unit 360 is electrically connected with the control unit 600 of the temperature adjusting part.

If the internal temperature value of the plant space part 1 does not reach the preset reference temperature value, the control unit 600 controls the operations of the open/close units 500 and the heating unit 400 as described above and transmits the electrical signal to the water wheel control unit 360.

The water wheel control unit 360 controls the operation of the rotational motor 350 so that the revolutions of the rotational shaft 320 are increased to a predetermined value or more. In this case, the rotational speed of the first and second blades 330 and 340 formed on the rotational shaft 320 are increased.

Accordingly, the flow rate of the fluid circulating along the circulation flow path a of the seawater stored in the storage space of the water tank body 210 may be increased. In addition, since the number of times of dropping the seawater is increased, an amount of oxygen generated in the seawater is increased.

According to the present invention, if the internal temperature of the plant space part 1 is decreased to a predetermined temperature or less, the circulation flow rate of the seawater is increased, so that the activity of the microalgae can be forcibly increased.

Meanwhile, the temperature adjusting part may control the operation of the lighting part 4 shown in FIG. 4.

The lighting control unit 730 is electrically connected with the control unit 600 of the temperature adjusting part.

If the internal temperature value of the plant space part 1 does not reach the preset reference temperature value range, the control unit 600 controls the operations of the open/close units 500 and the heating unit 400 as described above and transmits the electrical signal to the lighting control unit 730.

The lighting control unit 730 may increase illuminance to a predetermined level.

Therefore, the seawater stored in the storage space of the water tank body 210 is exposed to the light emitted with the increased illuminance. The microalgae included in the seawater exposed to the light actively perform photosynthesis.

In addition, since the light emitted according to the increase of the illuminance includes heat, the heat is transmitted to the seawater. Accordingly, the seawater may be increased to a predetermined temperature.

The seawater including the microalgae stored in the water tank body 210 is circulated within the water tank body 210 to receive oxygen.

In addition, the internal temperature value of the plant space part 1 having the water tanks 200 installed therein is controlled in real time in such a manner that the internal temperature value is included in the preset reference temperature value range.

Therefore, according to the present invention, the microalgae can be grown in large amounts by easily adjusting the growing environment of the microalgae.

The seawater including the microalgae grown in the water tanks 200 is discharged to the centrifuge 800 through the pipe.

The centrifuge 800 separates the material including microalgae from the seawater by using the difference in specific gravity between the material including the microalgae and the seawater. The material including the microalgae is a source material used to produce biofuel.

The source material is discharged to the oil press 810 through the pipe.

The oil press 810 destroys the cell wall of the microalgae source material to separate the oil component including the microalgae source material from other components, so that only the oil component is pressed. The oil press 810 may include another device and another method capable of separating oil components.

The oil component, which is pressed as described above, is discharged to an additional storage unit 820 and stored in the additional storage unit 820.

### INDUSTRIAL APPLICABILITY

Through the above-described operation, the present invention has an advantage of easily controlling growing conditions, such as a culture temperature and photosynthesis, capable of culturing microalgae in large amounts.

In addition, the present invention has an advantage of easily growing microalgae by continuously circulating the microalgae in a confined space and continuously supplying oxygen to the microalgae.

1. A plant for producing microalgae biofuel, the plant comprising:
a plant space part having an internal space;
a culture part provided in the internal space of the plant space part to culture microalgae by continuously circulating a fluid, which includes the microalgae and is supplied from an outside, at positions different from each other; and
a temperature adjusting part to adjust a temperature value of the internal space in the plant space part to a temperature value in a preset temperature value range.

2. The plant of claim 1, wherein the plant space part comprises a bottom part comprising concrete, a wall part surrounding edges of the bottom part, a ceiling part to cover an upper portion of the plant space part to form the internal space from the edges of the wall part, and one or more doors installed in the wall part to open/close the internal space, and each of the wall part, the ceiling part, and the doors comprises lattice frames comprising aluminum (Al) and glass plates installed between the lattice frames.

3. The plant of claim 2, wherein the culture part comprises a water tank provided in the internal space of the plant space part to store a predetermined amount of a fluid and having a circulation flow path of the fluid formed therein; and
a water wheel provided in the water tank and rotated by receiving power from an outside to forcibly move the fluid along the circulation flow path.

4. The plant of claim 3, wherein a lower portion of the water tank is inserted into the bottom part.

5. The plant of claim 3, wherein the water tank comprises a water tank body having an open upper portion and having a storage space in which the fluid is stored; and
a separator protruding upward from an internal floor of the water tank body to form the circulation flow path.

6. The plant of claim 5, wherein inclined surfaces are formed on a lateral side of the storage space in the water tank body and an outer surface of the separator to guide an interval between the lateral side of the storage space of the water tank body and the outer surface of the separator such that the interval is gradually narrowed.

7. The plant of claim 5, further comprising fluid confinement grooves formed in a lateral side of the storage space of the water tank body and the separator to limit a storage level of the fluid.

8. The plant of claim 5, wherein the water wheel comprises:
a support member selectively fixed to the water tank body such that the support member is provided on the circulation flow path;
a rotational shaft having both end portions rotatably supported on the support member such that the rotational shaft receives power from an outside to rotate;
first blades having a plate shape and installed radially on the rotational shaft;
second blades installed on end portions of the first blades and inclined at a predetermined angle with respect to each first blade;
a rotational motor linked with the rotational shaft to rotate the rotational shaft; and
a water wheel control unit to control an operation of the rotational motor.

9. The plant of claim 8, wherein the water wheel control unit receives an electrical signal from the temperature adjusting part to variably adjust a rotational speed of the rotational shaft based on the temperature value.

10. The plant of claim 1, wherein the temperature adjusting part comprises:
a heating unit installed on a floor of the culture part to heat the floor to a predetermined temperature;
one or more open/close units to receive an electrical signal from an outside to open/close the internal space of the plant space part;
a temperature sensor to measure an internal temperature value of the plant space part; and
a control unit to control operations of the heating unit and the open/close unit such that the measured temperature value by the temperature sensor is in the preset reference temperature value range.

11. The plant of claim 10, wherein the open/close unit comprises:
an open door rotatably supported on the plant space part to open/close an inner part of the plant space part;
a motor installed in the plant space part, and having a motor shaft to receive a control signal from the control unit to rotate;
a gear linked with the motor shaft; and
a rack protruding from the open door and engaged with the gear along a path having a predetermined curvature to rotate the open door according to a rotational motion of the motor shaft.

12. The plant of claim 10, wherein the heating unit comprises:
circulation pipes buried in the floor of the culture part while being spaced apart from each other by a predetermined distance; and
a boiler linked with the circulation pipes to heat heating water to a predetermined temperature by receiving an electrical signal from the control unit and to supply the heating water to the circulation pipes such that the heating water is circulated.

13. The plant of claim 1, further comprising a lighting part installed in the plant space part,
wherein the lighting part comprises: a light emitting device provided at an upper portion of the culture part to receive an electrical signal from an outside and to form predetermined illuminance; and
a lighting control unit electrically connected with the light emitting device to transmit a signal to the light emitting device such that preset illuminance and preset time to emit light are accomplished.

14. A bioreactor for producing biofuel, the bioreactor comprising:
a culture part having an open upper portion and formed therein with a circulation path of a fluid to produce the biofuel; and
a fluid moving part provided on the circulation part to forcibly circulate the fluid along the circulation path and to supply oxygen to the fluid.

15. The bioreactor of claim 14, wherein the culture part comprises:
sidewalls to form a storage space in which the fluid is stored;
a floor linked with lower end portions of the sidewalls; and
a separator installed at a central portion of the floor at a right angle to form the circulation path by dividing the storage space.

16. The bioreactor of claim 15, wherein a width of the circulation path when viewed in a sectional view is gradually narrowed downward from an upper portion of the culture part.

17. The bioreactor of claim 16, further comprising inclined surfaces formed on opposite surfaces of the sidewall and the separator to guide the opposite surfaces such that an interval between the opposite surfaces is gradually narrowed downward from the upper portion of the culture part.

18. The bioreactor of claim 14, wherein a lower portion of the culture part is inserted into a ground at a predetermined depth.

19. The bioreactor of claim 15, further comprising fluid level confinement grooves formed in the sidewalls and the separator to limit a storage level of the fluid.

20. The bioreactor of claim 14, wherein the fluid moving part comprises:
a support member installed in the culture part and having a rotational shaft;
blades provided in a plate shape, radially formed from an outer circumference of the rotational shaft, and having an end portion bent at a predetermined angle to move the fluid;
a rotational motor coupled with the rotational shaft to receive an electrical signal from an outside and rotate the rotational shaft; and
a fluid moving part control unit electrically connected with the rotational motor to control an operation of the rotational motor.

21. The bioreactor of claim 20, wherein each blade comprises:
a first blade provided in the plate shape and radially installed from the outer circumference of the rotational shaft; and
a second blade provided in the plate shape and installed on an end portion of the first blade such that the second blade is inclined at a predetermined angle with respect to the first blade.

22. The bioreactor of claim 20, further comprising a support frame between the rotational shaft and the blade, wherein the support frame comprises:
first support frames radially provided from a plurality of positions of an outer circumference of the rotational shaft while extending by a predetermined length; and
a second support frame linking end portions of the first support frames with each other along the circumference of the rotational shaft, each blade being fixed to the second support frame.

23. A method of producing microalgae biofuel, the method comprising:
storing a fluid comprising the microalgae in a bioreactor which has a circulation flow path formed therein and is provided in a plant;
continuously circulating the fluid along the circulation flow path;
adjusting a temperature value of an internal space in the plant to a temperature value in a preset temperature value range, or controlling a temperature value of the bioreactor according to the temperature value of the internal space in the plant; and
separating microalgae from the fluid and pressing an oil.

24. The method of claim 23, wherein the circulating of the fluid along the circulation flow path comprises:
preparing a forcible circulator, which forcibly forms a flow on the circulation path, in the bioreactor; and
forming continuous and forcible flow of the fluid along the circulation flow path by actuating the forcible circulator.

25. The method of claim 23, wherein the bioreactor is provided therein with a separator to form the circulation flow path,
level confinement grooves are formed in sidewalls and the separator of the bioreactor, and
the fluid is stored in the bioreactor by forming a level of the fluid to the level confinement grooves.

26. The method of claim 24, wherein the continuously-circulating of the fluid along the circulation flow path comprises controlling an operation of the forcible circulator such that a forcible flow speed is varied depending on the temperature value of the internal space in the plant or the temperature value of the fluid stored in the bioreactor.

27. The method of claim 23, wherein the adjusting of the temperature value of the internal space in the plant comprises:
selecting one of the temperature value of the bioreactor and the temperature value of the internal space in the plant to be controlled;
adjusting a temperature of the bioreactor depending on the temperature value of the internal space in the plant if the temperature value of the bioreactor is selected to be controlled; and
adjusting ventilation in the plant such that the temperature value of the internal space in the plant is in the preset temperature value range if the temperature value of the internal space in the plant is selected to be controlled.

28. The method of claim 27, wherein the adjusting of the temperature of the bioreactor comprising:
measuring the temperature value of the internal space in the plant by using a temperature sensor;
transmitting the measured temperature value of the internal space in the plant to a control unit; and
hating a floor of the bioreactor to a predetermined temperature by actuating a heating unit installed in the floor of the bioreactor through the control unit, such that the temperature value measured by the temperature sensor is in a range of a preset first reference temperature value range.

29. The method of claim 28, wherein a circulation pipe is buried in the floor of the bioreactor, heating water is heated to a predetermined temperature by using a boiler which receives an electrical signal from the control unit, and the heating water, which is heated, is supplied into the circulation pipe and circulated to heat the floor of the bioreactor.

30. The method of claim 27, wherein the temperature value of the internal space in the plant is measured by using a temperature sensor and transmitted to a control unit, and
the internal space in the plant is open/closed by actuating one or a plurality of ventilation adjusting units using the control unit such that the temperature value measured by the temperature sensor is in a preset second reference temperature value range.

31. The method of claim 23, further comprising constantly providing light to the fluid circulated along the circulation flow path, wherein the constantly providing of the light comprises:
arranging lamps on an upper portion of the bioreactor such that the lamps receive an electrical signal from an outside and emit light with predetermined illuminance; and
accomplishing preset illuminance and preset time to emit light by using a light control unit.
